(19) United States
(12) Patent Application Publication
(10) Pub. No.: US 2024/0057927 A1
(43) Pub. Date: Feb. 22, 2024

(54) SYSTEMS AND METHODS FOR MEASURING NEUROLOGIC FUNCTION VIA ODORANT, AUDIBLE AND/OR SOMATOSENSORY STIMULATION

(71) Applicant: OLFAXIS, LLC, Durham, NC (US)

(72) Inventors: David William SMITH, Durham, NC (US); Richard MICHELLI, Raleigh, NC (US); William David WOOLF, Durham, NC (US)

(21) Appl. No.: 18/470,258

(22) Filed: Sep. 19, 2023

Related U.S. Application Data
(62) Division of application No. 16/977,597, filed on Sep. 2, 2020.
(60) Provisional application No. 62/640,364, filed on Mar. 8, 2018.

(51) Int. Cl. A61B 5/381; A61B 5/38; A61B 5/291

(52) U.S. Cl. CPC A61B 5/381; A61B 5/38; A61B 5/291; A61B 5/6819

(57) ABSTRACT

A system for evaluating neurologic dysfunction of a subject includes an odorant generator configured to deliver an odorant stimulation to the subject, an auditory generator configured to deliver an audible stimulation to the subject, a vibrotactile stimulator configured to generate a somatosensory stimulation to the subject, a plurality of electrodes configured to be attached to the subject at respective different locations, and at least one processor. The plurality of electrodes are configured to collect neural signals from the subject as a result of the odorant stimulation, the audible stimulation, and the somatosensory stimulation. The at least one processor is configured to process the neural signals from the plurality of electrodes and generate an assessment of neurologic dysfunction of the subject.

### RELATED APPLICATIONS

This application is a divisional application of pending U.S. patent application Ser. No. 16/977,597, filed Sep. 2, 2020, which is a 35 U.S.C. § 371 national stage application of PCT Application No. PCT/US2019/021094, filed on Mar. 7, 2019, which claims the benefit of and priority to U.S. Provisional Patent Application No. 62/640,364 filed Mar. 8, 2018, the disclosures of which are incorporated herein by reference as if set forth in their entireties. The above-referenced PCT International Application was published in the English language as International Publication No. WO 2019/173558 A1 on Sep. 12, 2019.

### FIELD OF THE INVENTION

The present invention relates generally to systems and methods of evaluating neurologic dysfunction.

### BACKGROUND OF THE INVENTION

There is significant evidence that the sense of smell is disrupted by brain dysfunction; changes in smell are some of the best predictors of mild traumatic brain injury (mTBI) and neurodegenerative diseases (e.g., Alzheimer's and Parkinson's Diseases). Changes in smell are sensitive indicators of mTBI, even in the absence of radiographic evidence of injury.

Most of the extant scientific literature supporting the link between olfactory deficits and mTBI/neurodegenerative diseases is derived from behavioral/perceptual olfactometry studies—at present the gold standard. In some patients, however, behavioral smell tests are not possible (i.e., the patient is unconscious, uncooperative or an infant). In these subjects, electrophysiological measures may be the best alternative, measures comparable to otoacoustic emissions and/or ABR tests of hearing.

Neurological measures of olfactory function (olfactory evoked potentials (OEPs) and olfactory event-related potentials (OERPs)), which can be measured using quantitative electroencephalographic (qEEG) techniques, are highly correlated with the behavioral measures but are less frequently used and therefore less understood as indicators of mTBI and neurodegenerative diseases. OEPs and OERPs can be measured using scalp EEG electrodes. Using standard EEG methods, it is also possible to simultaneously visualize cortical alpha band oscillations along with the OEPs and OERPs. Alpha band oscillations are generated by thalamic pacemaker cells and are present when the brain is unstimulated (i.e., is “idling”) and are believed to aid in detecting new, incoming sensory stimulation; alpha oscillations rapidly decrease when the brain is activated by external sensory stimuli.

There remains a need for systems and methods that provide measures of the conduction of neural information from sensory receptors in the nose through diffuse projections within the brain.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

Embodiments of the present invention include systems and methods that use olfactory stimulation, through natural sensory receptors and neural pathways, to generate OEPs and OERPs (and to suppress alpha band oscillations) in conjunction with multimodal assessment using somatosensory and/or auditory stimulation. Changes in olfactory function are sensitive indicators of neurological function in and of themselves; however, by combining olfactory, somatosensory, and auditory measures, this approach provides a novel and powerful electrophysiological measure of brain neural function for use in detecting mTBI and/or neurodegenerative diseases, like Parkinson's and or Alzheimer's disease, that does not require behavioral responding from the test subject.

In some embodiments, the systems include an intranasal delivery apparatus that is a handheld device. In other embodiments, the intranasal delivery apparatus is supported by a stand.

According to some embodiments of the present invention, a system for measuring olfactory evoked potentials includes an air (or other gas) source (e.g., an air pump or pressurized air source) configured to provide a first stream of clean, odorless control air, an odorant generator configured to generate a second stream of odorized air, and an intranasal delivery system. A first valve is coupled to the air source and to the intranasal delivery assembly, a second valve is coupled to the odorant generator and to the intranasal delivery assembly, and a controller is coupled to the first and second valves. The controller is configured to direct the first and second valves to selectively open and close such that the first stream of odorless control air and the second stream of odorized air can be selectively directed to the intranasal delivery assembly to deliver an odorant stimulation to the subject via the intranasal delivery assembly.

The odorant generator is configured to generate the second stream of odorized air with a defined odorant concentration. In some embodiments, the odorant generator is configured to generate the second stream of odorized air with a selected one of a plurality of different odorant concentrations.

The controller is configured to direct the first and second valves to selectively open and close such that the odorant stimulation has an abrupt onset. The controller is also configured to direct the first and second valves to selectively open and close such that there is no perceptible disturbance of air flow to the subject.

In some embodiments, the intranasal delivery assembly includes a first tube connected to the first valve, a second tube connected to the second valve, a third tube in fluid communication with the first and second tubes via a first connector (e.g., a Y-connector, etc.), and first and second delivery tubes. Each delivery tube includes a proximal end and an opposite distal end, and the proximal end of each delivery tube is in fluid communication with the third tube via a second connector (e.g., a Y-connector, etc.). A bung is secured to the distal end of each delivery tube, and each bung is configured to be inserted into a respective nostril of the subject. In some embodiments, each bung has a generally cylindrical body with electrically conductive material, such as foil, attached to an outer surface of the body.

In some embodiments, the system also includes an auditory sound generator and communicates with the controller and delivers sounds through a transducer such as an earbud insert earphone, etc.

In some embodiments, the system also includes a somatosensory stimulator that communicates with the controller and delivers vibratory stimuli or electrical stimuli to the skin through a vibrotactile stimulator or skin electrodes that can be affixed to the hand, arm, leg, torso, or other body part.

In some embodiments, the system also includes a plurality of electrodes configured to be attached to the subject at respective different locations. Each electrode is configured to collect neural signals from the olfactory epithelium or different cortical areas in the brain of the subject. The system also includes a signal processor configured to receive and process the neural signals from the plurality of electrodes, and a signal amplifier configured to receive and amplify the neural signals from the plurality of electrodes prior to processing by the signal processor.

In some embodiments, the odorant generator includes an odorant cartridge configured to aerosolize a liquid odorant contained therewithin. The cartridge may include a frangible container of the liquid odorant and a plunger configured to break the frangible container to release the liquid odorant.

According to some embodiments of the present invention, a system for measuring neurologic function of a subject includes an odorant generator configured to deliver an odorant stimulation to the subject, an auditory generator configured to deliver an audible stimulation to the subject, and at least one electrode configured to be attached to the subject. The at least one electrode is configured to collect neural signals from the subject as a result of the odorant stimulation and the audible stimulation. The at least one electrode may include a plurality of electrodes configured to be attached to the subject at respective different locations. The system further includes at least one processor configured to process the neural signals from the at least one electrode and generate an assessment of the neurologic function of the subject.

In some embodiments, the auditory generator is configured to deliver an audible stimulation to the subject via one or more earbuds worn by the subject. However, other types of audio devices may be utilized.

In some embodiments, the system may also include a vibrotactile stimulator configured to generate a somatosensory stimulation to the subject. For example, the vibrotactile stimulator may be configured to generate a somatosensory stimulation to skin of the subject. The at least one electrode is configured to collect neural signals from the subject as a result of the somatosensory stimulation. In some embodiments, somatosensory stimulation may be generated via electrical stimulation, such as electrodes attached to the skin of the subject.

In some embodiments, the odorant generator is a handheld intranasal delivery assembly. In other embodiments, the odorant generator comprises a mask configured to be placed over a face of the subject, such as nonresponsive (e.g., loss of consciousness) or uncooperative subjects (e.g., malingers or infants).

According to other embodiments of the present invention, a system for measuring neurologic function of a subject includes, an odorant generator configured to deliver an odorant stimulation to the subject, an auditory generator configured to deliver an audible stimulation to the subject, a vibrotactile stimulator configured to generate a somatosensory stimulation to the subject, a plurality of electrodes configured to be attached to the subject at respective different locations, and at least one processor. The plurality of electrodes are configured to collect neural signals from the subject as a result of the odorant stimulation, the audible stimulation, and the somatosensory stimulation. The at least one processor is configured to process the neural signals from the plurality of electrodes and generate an assessment of neurologic function of the subject.

According to other embodiments of the present invention, a method of measuring neurologic function of a subject includes delivering an odorant stimulation to the subject, delivering an audible stimulation to the subject, delivering a somatosensory stimulation to the subject, collecting neural signals from the subject via one or more electrodes attached to the subject as a result of the odorant stimulation, the audible stimulation, and the somatosensory stimulation, and processing the neural signals via at least one processor to generate an assessment of neurologic function of the subject. In some embodiments, the odorant stimulation, the audible stimulation, and the somatosensory stimulation are delivered to the subject at substantially the same time. An assessment of neurologic dysfunction of the subject, such as from mTBI or a concussion, can then be determined by comparing the generated neurologic function assessment to a baseline of neurologic function for the subject.

In some embodiments, the odorant stimulation, the audible stimulation, and the somatosensory stimulation are delivered to the subject sequentially. In some embodiments, the audible stimulation and the somatosensory stimulation are delivered to the subject before the odorant stimulation. In some embodiments, the audible stimulation and the somatosensory stimulation are delivered to the subject after the odorant stimulation.

Embodiments of the present invention are advantageous because OEPs can be measured in uncooperative (e.g., infants or malingers) or unconscious subjects.

Embodiments of the present invention are also advantageous because OERPs can be measured. OERPs are responses of cortical and higher level neurons to olfactory stimulation. The presence of OERPs can also be verified as changes in cortical alpha band oscillations, and changes in alpha band oscillations produced by sensory stimulation, including by odorant stimulation, have been shown to reflect mTBI. By using OERPs, it may be possible to assess higher level, cognitive function/dysfunction. Inclusion of auditory and somatosensory stimulation, either before, simultaneous with, or following odorant stimulation will allow assessment of function in wider brain regions. Embodiments of the present invention are advantageous because, by integrating multisensory stimulation, a more comprehensive assessment of brain function to diagnose and monitor mTBI can be obtained.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The accompanying drawings, which form a part of the specification, illustrate various embodiments of the present invention. The drawings and description together serve to fully explain embodiments of the present invention.

FIG. 1 illustrates a system for measuring OEPs, OERPs, auditory, and somatosensory evoked potentials nearly simultaneously in a single test session, according to some embodiments of the present invention.

FIG. 2 illustrates a portion of an intranasal delivery system that is part of the system of FIG. 1, according to some embodiments of the present invention.

FIG. 3 illustrates the intranasal delivery system of FIG. 2 attached to a subject for the measurement of OEPs/OERPs.

FIG. 4A is a schematic diagram of the valve assembly and the intranasal delivery system of FIG. 2, according to some embodiments.

FIG. 4B is a schematic diagram of the valve assembly and the intranasal delivery system of FIG. 2, according to other embodiments.

FIG. 5 is a schematic diagram of an odorant generator that can be utilized with the system of FIG. 1, according to some embodiments of the present invention.

FIG. 6 is an illustration of an example scalp map (e.g., the international standard 10-20 map) of EEG electrode placements used to collect neural signals in response to olfactory, auditory, and somatosensory stimuli according to some embodiments of the present invention. Active sensors are indicated in red, ground sensors in green (A1 for OEP, Pz for OERP) and reference sensor as yellow.

FIGS. 7A-7B are schematic diagrams illustrating the use of a first stream of odorless control air and a second stream of odorized air in accordance with embodiments of the present invention. FIG. 7A illustrates an interstimulus interval where a subject's nose receives the first stream of odorless control air only, and the second stream of odorized air is directed towards a vacuum line. FIG. 7B illustrates a stimulation interval where the second stream of odorized air is briefly directed to the subject's nose, and the first stream of odorless control air is redirected towards the vacuum line.

FIG. 8 illustrates an exemplary square wave form of air and odorant flows where an airstream containing an odorant is inserted into an airstream of odorless air.

FIGS. 9A-9B illustrate an odorant cartridge that may be utilized with the odorant generator of the system of FIG. 1, according to some embodiments of the present invention.

FIG. 9C illustrates plungers of an odorant cartridge that are configured to mechanically break glass ampoules within the cartridge to release liquid phase odorant onto the absorbent material in the interior of the cartridge, according to some embodiments of the present invention.

FIG. 10 is a perspective view of a bung that can be utilized with the intranasal delivery assembly of FIG. 2, according to some embodiments of the present invention.

FIG. 11A illustrates a graph of odorant concentration vs. time of a “staircase” procedure that may be utilized to estimate the neural threshold for eliciting an OEP from a subject, showing changes in evoked potential amplitude with increases in stimulus intensity.

FIG. 11B illustrates a graph of amplitude vs. odorant concentration with hypothetical (prophetic) changes in neural responding with odorant concentration.

FIGS. 12A-12D illustrate graphs of mV vs. time (ms) of exemplary OEP individual wave form data obtained using the system of FIG. 1.

FIGS. 13A-13C illustrate color-coded exemplary OERP individual topographical heat map data using the system of FIG. 1.

FIG. 14 illustrates exemplary alpha wave oscillation data using the system of FIG. 1.

FIGS. 15A-15N illustrate exemplary alpha wave spectra grand mean data from a group of simulated subjects tested using the system of FIG. 1.

FIGS. 16A-16B illustrate exemplary alpha band suppression grand mean data from a group of hypothetical subjects tested using the system of FIG. 1.

FIGS. 17A-17F illustrate exemplary multisensory individual data using the system of FIG. 1.

FIG. 18 is a flowchart that illustrates methods of evaluating neurologic dysfunction via odorant, audible and/or somatosensory stimulation, according to some embodiments of the present invention.

### DETAILED DESCRIPTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. In the figures, certain layers, components or features may be exaggerated for clarity, and broken lines illustrate optional features or operations unless specified otherwise. In addition, the sequence of operations (or steps) is not limited to the order presented in the figures and/or claims unless specifically indicated otherwise. Features described with respect to one figure or embodiment can be associated with another embodiment or figure although not specifically described or shown as such.

It will be understood that when a feature or element is referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “secured”, “connected”, “attached” or “coupled” to another feature or element, it can be directly secured, directly connected, attached or coupled to the other feature or element or intervening features or elements may be present. In contrast, when a feature or element is referred to as being, for example, “directly secured”, “directly connected”, “directly attached” or “directly coupled” to another feature or element, there are no intervening features or elements present. The phrase “in communication with” refers to direct and indirect communication. Although described or shown with respect to one embodiment, the features and elements so described or shown can apply to other embodiments.

The term “circuit” refers to software embodiments or embodiments combining software and hardware aspects, features and/or components, including, for example, at least one processor and software associated therewith embedded therein and/or executable by and/or one or more Application Specific Integrated Circuits (ASICs), for programmatically directing and/or performing certain described actions, operations or method steps. The circuit can reside in one location or multiple locations, it may be integrated into one component or may be distributed, e.g., it may reside entirely or partially in a portable housing, a workstation, a computer, a pervasive computing device such as a smartphone, laptop or electronic notebook, or partially or totally in a remote location away from a local computer or processor of a respective test unit or device or a pervasive computing device such as a smartphone, laptop or electronic notebook. If the latter, a local computer and/or processor can communicate over local area networks (LAN), wide area networks (WAN) and can include a private intranet and/or the public Internet (also known as the World Wide Web or “the web” or “the Internet”). Systems and devices according to embodiments of the present invention can comprise appropriate firewalls and electronic data interchange standards for HIPPA or other regulatory compliance. In the traditional model of computing, both data and software are typically substantially or fully contained on the user's computer; in cloud computing, the user's computer may contain little software or data (perhaps an operating system and/or web browser), and may serve as little more than a display terminal for processes occurring on a network of external computers. A cloud computing service (or an aggregation of multiple cloud resources) may be generally referred to as the “Cloud”. Cloud storage may include a model of networked computer data storage where data is stored on multiple virtual servers, rather than being hosted on one or more dedicated servers. Data obtained by various systems and devices according to embodiments of the present invention can use the Cloud.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.”, which derives from the Latin phrase “exempli gratia,” may be used to introduce or specify a general example or examples of a previously mentioned item, and is not intended to be limiting of such item. The common abbreviation “i.e.”, which derives from the Latin phrase “id est,” may be used to specify a particular item from a more general recitation.

As used herein, the term “and/or” includes any and all combinations of one or more of the associated listed items and may be abbreviated as “/”.

As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that although the terms first and second are used herein to describe various features or elements, these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The term “about”, as used herein with respect to a value or number, means that the value or number can vary by +/−twenty percent (20%).

Olfactory neural pathways, originating in the nasal cavity, reach into the central nervous system where they branch diffusely within the brain; these tracts play critical roles in the brain's most important functions, including emotion, memory and executive function. As a consequence, damage to any of these areas can result in changes in cognitive, emotional and olfactory function (cf., Osborne-Crowley, 2016; Alosco et al., 2016). Research studies have repeatedly shown a relationship between olfactory dysfunction and traumatic brain injury (TBI) (Frasnelli et al., 2015; Caminiti et al., 2013; Drummond et al., 2015). Likewise, it is known that changes in olfactory function are some of the first, and most accurate predictors of the eventual onset of Parkinson's and Alzheimer's Diseases (cf., Doty, 2003; Berendse et al., 2011; Doty, 2012; Rahayel et al, 2012; Velayudhan et al., 2013; Behrman et al., 2014). The contents of these documents are hereby incorporated by reference as if recited in full herein. TBI is one of the most common causes of olfactory dysfunction, though most of the afflicted are unaware of the sensory deficit.

The term “olfactory evoked potential” (OEP) refers to the electrical neural responses generated by the response (neural receptor potentials) of olfactory receptors (in, and between, the main olfactory epithelium in the nasal cavity and the olfactory bulb in the forebrain) to odorant stimulation. OEPs can be obtained using an electrode placed in the epithelium, nasal cavity, on the surface of the bridge of the nose, or on the scalp. The term “olfactory event related potential” (OERP) refers to the electrical neural responses generated in cortical neurons by neural electrical activity conducted from “lower” regions of the olfactory central nervous system (i.e., olfactory receptors and olfactory bulb). OERPs can be obtained from surface electrodes using standard electroencephalographic (EEG) electrodes, methods and instrumentation.

A stimulus for evoking any neural evoked potential, whether it is olfactory, auditory, visual or somatosensory, is preferably a stimulus with an abrupt onset. A preferred odorant stimulus for evoking sensory evoked potentials can have an infinite rise time and offset—a perfect square wave.

The neurophysiological reason for the stimulus is that the neural response from any one receptor is so small that it may not be seen above normal background physiological noise created by muscles, eye movement, etc. Therefore, to visualize the neural response above the background noise, one needs to see the summed activity of many olfactory receptors activated at precisely the same moment—then improve that by using signal averaging to increase the signal-to-noise ratio. Therefore, an odorant/stimulus delivery with as close to an instantaneous onset and offset is accomplished by embodiments of the present invention.

There is significant evidence that the sense of smell is disrupted by head trauma, and that changes in smell are some of the best predictors of TBI. Changes in smell are sensitive indicators of TBI, even in the absence of radiographic evidence. Changes in olfactory function are sensitive indicators of neurodegenerative diseases; because of the sensitivity, some have argued that Parkinson's Disease is an olfactory disease. Most of the data in the scientific literature supporting the link between olfactory deficits and TBI and neurodegenerative diseases are from behavioral/perceptual olfactometry studies. Electrophysiological measures of olfactory function (OEPs and OERPs) are highly correlated with the behavioral measures, but are less frequently used and, therefore, less understood as indicators of neurologic dysfunction. The current scientific literature also suggests that the degree of olfactory dysfunction following head trauma predicts/indicates the magnitude and, possibly, the location of TBI. These data are primarily from behavioral measures.

Embodiments of the invention can provide olfactory function tests that have clinical utility and may be used for patient screening, i.e., to deliver results that inform decisions about treatment of patients, potentially in conjunction with other testing. Embodiments of the invention can use evaluation of olfactory function to assess whether a patient/user may have TBI. Degradation of olfactory function can also be a biomarker for other neurological conditions and neurodegenerative diseases.

Additional embodiments of the present invention can use multisensory stimulation, where auditory sounds and somatosensory vibrotactile stimuli are presented before, simultaneous with, or after the odorant. Multisensory stimulation will activate and assess function in wider brain regions than olfactory stimulation alone.

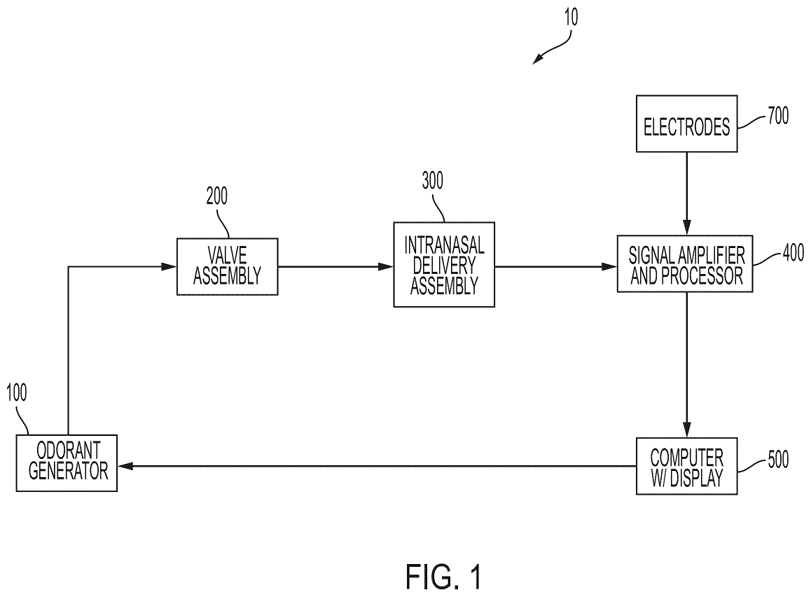

FIG. 1 illustrates a system 10 for measuring OEP and OERP multimodal responses, according to some embodiments of the present invention, and that can be utilized with auditory and somatosensory stimuli generating devices (described below). The system 10 allows multisensory collection on cooperative as well as nonresponsive (e.g., loss of consciousness) or uncooperative subjects (e.g., malingers or infants). The system 10 includes an odorant generator 100, a valve assembly 200 for controlling delivery of an odorant (e.g., phenyl ethanol, butanol, propanol, cinnamon, etc.), an intranasal delivery assembly 300 for delivering an odorant to a subject, a signal amplifier and processor 400, a computer with display 500. The odorant generator 100, described further below, provides an odorant pulse to the valve assembly 200 and intranasal delivery assembly 300. The intranasal delivery assembly 300, in addition to delivering an odorant to a subject, includes surface recording electrodes that collect neural signals from the olfactory epithelium of the subject.

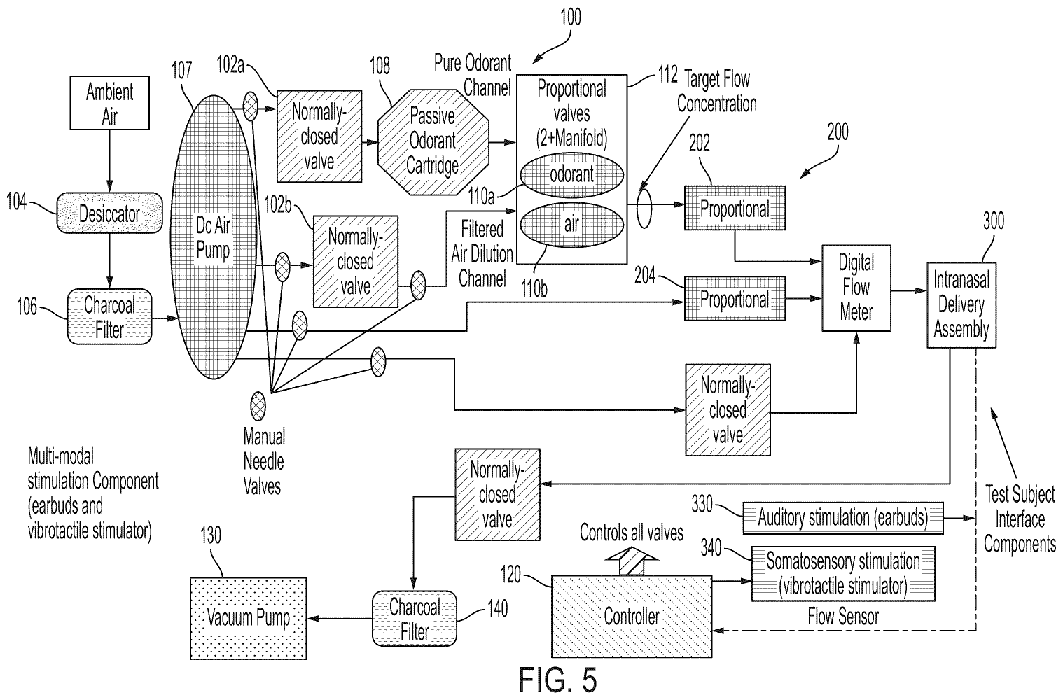

The illustrated system 10 may be utilized with devices for generating auditory and somatosensory stimuli, as described with respect to FIG. 5. For example, sounds are generated by an audio source and communicated to a person's ear via inputs 330, such as earbuds/earphones or via an acoustic transducer. The controller 120 can also direct one or more somatosensory generators to generate somatosensory stimuli to the skin of a person via activation of a vibrotactile stimulator 340 (FIG. 5).

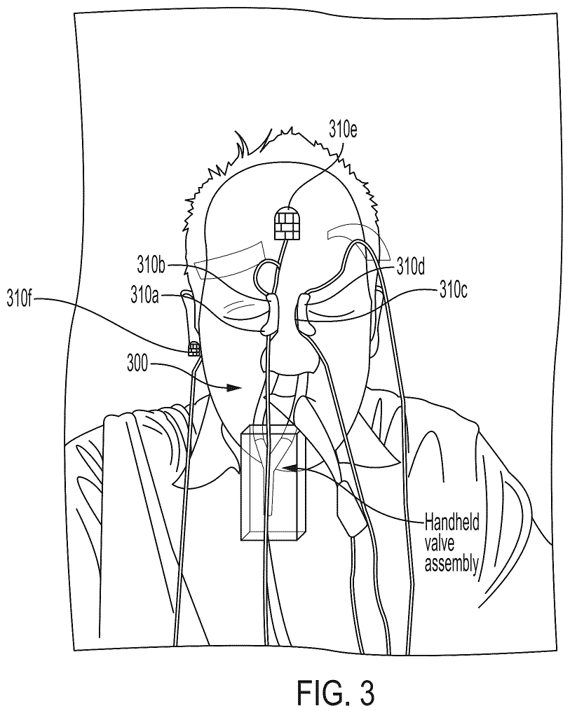

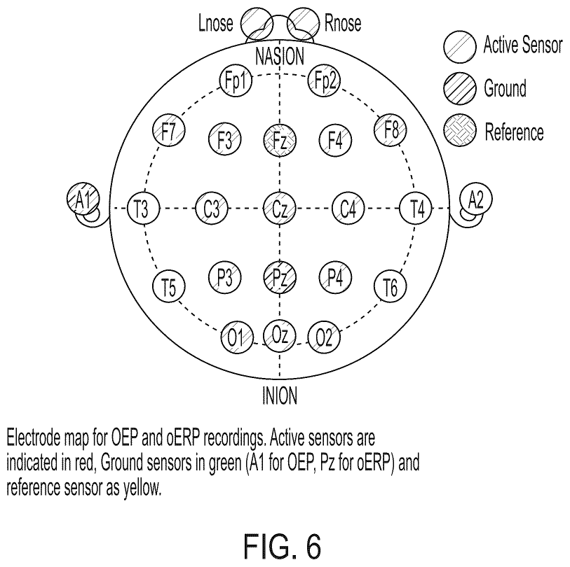

Neural signals collected by various electrodes attached to a person, for example as shown in the electrode map 700 (FIG. 6), are sent to individual channels on the amplifier/processor 400 for amplification. In addition, the amplifier/processor 400 may be configured to increase the signal integrity of collected neural signals. The amplifier/processor 400 may be an independent unit, as illustrated, or may be integrated into the electrodes 310a-310f (FIG. 3). FIG. 6 illustrates a map 700 exemplary locations for electrodes. However, other electrode placement configurations and other numbers (i.e., 5-25, 10-20, etc.) of electrodes may be utilized in accordance with embodiments of the present invention.

The amplified neural signals are processed by the amplifier/processor 400 to aid in the identification of neural signals from noise. The amplifier/processor 400 can take inputs from a number of different channels/electrodes and, using digital signal processing, can filter and store neural responses from signals from the electrode map 700.

The processed digital signals are then sent to a computer 500 for averaging and formatting for display. The raw wave form data can be shown on the display and can be stored and further processed by the computer 500. The neural signals created when odorant, auditory, and somatosensory stimuli are applied are very small compared with the background physiological noise which are created, for example, by muscle artifacts, the movement of blood, respiration etc. A single response of a neuron, or even a group of neurons, can be obscured by such background noise. The computer 500 uses signal averaging software to display OEPs, OERPs, auditory and somatosensory responses. Signal averaging is a signal processing technique used to increase the strength of a signal relative to noise that is obscuring it. By averaging a set of replicate measurements, the signal-to-noise ratio (S/N) will be increased, and the noise will average to near zero (0), while the amplitude of the biological signal will be increased. The computer 500 can also be configured to control the overall test system 10, including delivery of multisensory stimuli to a subject via the various devices (i.e., odorant generator 100, auditory generator 330, somatosensory generator 340).

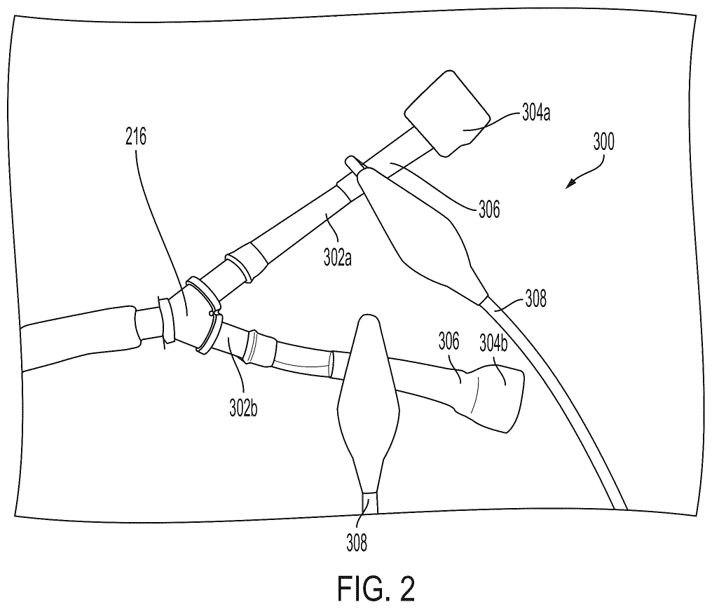

FIGS. 2, 3, 4A, 4B illustrate an intranasal delivery assembly 300 according to some embodiments of the present invention. The illustrated intranasal delivery assembly 300 is configured for birhinal odorant delivery and includes a plurality of electrodes 310a-310f (FIG. 3) and two delivery tubes 302a, 302b for simultaneous odorant delivery to both nostrils of a subject. At the end of each delivery tube 302a, 302b is a respective bung 304a, 304b comprising an electrically conductive outer layer such as conductive foil. Each bung 304a, 304b serves as an electrode for recording evoked potentials in a nasal cavity.

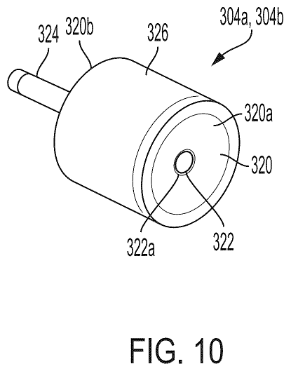

As illustrated in FIG. 10, each bung 304a, 304b has a body 320 with a generally cylindrical configuration. The body 320 may be formed from foam or other elastomeric materials. A passageway 322 extends through the body 320 and terminates at an aperture 322a in the distal end 320a of the body 320, as illustrated. A hollow tube 324 extends outwardly from the proximal end 320b of the body 320 and is in fluid communication with the passageway 322. The tube 324 is configured to be inserted within, or otherwise attached to, one of the delivery tubes 302a, 302b. Thus, when a bung 304a, 304b is attached to a respective delivery tube 302a, 302b, a generated odorant flows through the respective delivery tube 302a, 302b, through the hollow tube 324, through the passageway 322 in the body 320 of the bung 304a, 304b, and exits through the aperture 322a into a nostril of a subject.

As illustrated in FIG. 10, each electrode bung 304a, 304b is covered with a conductive foil 326. Exemplary conductive foil includes copper or gold foil. However, any conductive foil may be utilized. Conductive gel may be placed on the electrode bungs 304a, 304b, and the bungs 304a, 304b are inserted as far as comfortable, mono- or bi-rhinally into the nose of the subject, as illustrated in FIG. 3. Electrically conductive tape 306 (FIG. 2), such as copper tape, etc., on each bung 304a, 304b is used to secure wire leads 308 that are connected to the amplifier 400. The wire leads 308 may be electrically connected to the conductive tape via alligator clips, solder or the like.

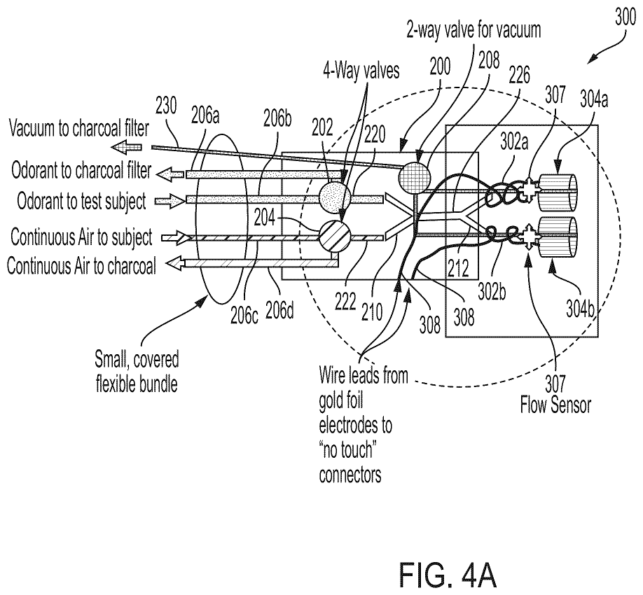

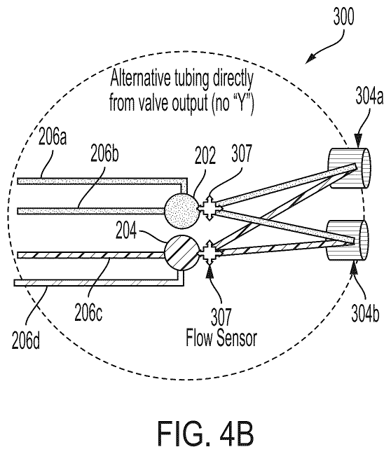

Flow sensors 307 (FIGS. 4A-4B) may be coupled to the bungs 304a, 304b to detect the exhalation/inhalation pattern of a subject and to allow fully automated control of the odorant pulse delivery during subject inhalations.

FIG. 3 illustrates the bungs 304a, 304b inserted within the nostrils of a subject and the electrodes 310a-310f attached to the subject at various locations. Typically, a subject will be supine with eyes closed, and the locations on the subject's face where the electrodes 310a-310f will be attached are scrubbed with disposable alcohol wipes to remove oils and dirt to improve conductivity and the attachment strength of the electrode adhesive. In the illustrated embodiment of FIG. 3, four pad electrodes 310a-310d, such as Red Dot™ brand EKG electrodes, available from 3M Company, Saint Paul, Minnesota, are attached bilaterally about 1 centimeter off the midline, just above and just below the medial canthus of the subject. A reference electrode 310e is placed on the forehead of the subject, and a ground electrode 310f is placed on the left ear of the subject. These electrodes 310a-310f are attached via lead wires to “no-touch” connectors on the valve assembly box 200. The illustrated intranasal delivery assembly 300 is designed for single use and is disposable.

A valve assembly device 200 (FIG. 1) is connected to the intranasal delivery assembly 300 and controls the delivery of odorant to the intranasal delivery assembly 300. In some embodiments, the valve assembly device 200 may be a structure formed to approximate the curvature of the human face and may be held against the face with elastic bands, etc. In other embodiments, the valve assembly device 200 may be a handheld device.

In other embodiments, the valve assembly device 200 may be supported by a stand or other structure and is not held by a person being tested. The valve assembly device 200 holds a series of miniature solenoid valves that produce electromechanical stimuli when activated. Use of a stand or other structure to hold the valve assembly device 200 can prevent inadvertent, uncontrolled mechanical and electrical stimulation of the hand and the mechanosensory neural system, which can result in unwanted cortical activity and confound interpretation of the desired odorant responses. In some embodiments, the system of the present invention is designed to introduce specified and controlled somatosensory stimulation using 0.2-2.0 ms square electrical pulses through surface electrodes on the median nerve at the wrist, or vibrotactile stimulation on the fingers. As such, a stand or other structure supporting the valve assembly device 200 can eliminate the possibility of the valves causing unwanted stimulus to the person, particularly where it is desired to introduce specified and controlled somatosensory stimulation and/or vibrotactile stimulation.

In other embodiments, the intranasal delivery assembly 300 may be configured as a mask that is placed over the face of a subject, such as a nonresponsive (e.g., loss of consciousness) subject or uncooperative subjects (e.g., malingers or infants).

Referring to FIG. 4A, the interconnection of the valve assembly 200 and the intranasal delivery assembly 300, according to some embodiments of the present invention, is illustrated. A continuous loop of a first stream of odorless filtered control air and a second stream of odorized air are delivered to separate valves 202, 204 in the valve assembly 200 through separate, dedicated lines. For example, the continuous loop of the first stream of odorless control air is delivered to the valve 204 from an air source (e.g., an air pump, compressed air source, etc.) via line 206c and returned to a charcoal filter via line 206d. The continuous loop of the second stream of odorized air is delivered to the valve 202 from an odorant generator via line 206b and returned to a charcoal filter via line 206a. In between odorant pulses, the continuous first stream of odorless control air is delivered to a subject via the intranasal assembly 300, while the second stream of odorized air is shunted by valve 202 to the charcoal filter.

To interconnect the valve assembly 200 and the intranasal delivery assembly 300, a first tube 220 is connected to the valve 202, a second tube 222 is connected to the valve 204, and a third tube 226 is in fluid communication with the first and second tubes 220, 222 via a first connector 210, such as a Y-connector. The first Y-connector 210 joins the outputs of the two valves 202, 204 into the third tube 226, and a second Y-connector 212 separates the airflow for odorant delivery to the two nostrils of the subject via the delivery tubes 302a, 302b of the intranasal delivery assembly 300.

At the initiation of a test sequence, valve 204 is fully open delivering a continuous, clean, filtered airstream to the test subject, valve 202 is closed blocking the flow of the odorized airstream. To deliver the test odorant, valve 204 is closed, thereby routing the clean airstream to the charcoal filter, at the same instant that valve 202 is opened to route the odorized airstream to the test subject via the intranasal assembly 300 or for the duration of the odorant pulse (e.g., 200 to 800 milliseconds, although other durations may be utilized). Another valve 208 is provided for controlling a vacuum line 230 and is activated after odorant delivery to evacuate residual odorized air from the nasal cavity. In some embodiments, the various tubes (e.g., delivery tubes 302a, 302b, lines 206a-206d and 226) have a minimum internal diameter (ID) of about 3/16″. However, embodiments of the present invention are not limited to tubes or connectors having a particular ID and/or configuration.

The system 10 of FIG. 1 is configured to automatically measure olfactory evoked potentials and trigeminal evoked potentials. Chemically-evoked trigeminal potentials are less, or not affected by neurological insult, so can be used to verify device function and ability to measure neural signal. If trigeminal OEP or OERP is the same, but olfactory (both measured in the same session with a subject) is decreased, stronger olfactory evidence of neurologic insult exists. If only using olfactory OEP and/or OERP measures and no neural signal observed, it may be difficult to determine if a subject's neural system is bad or if the recording instrumentation system was not working properly. Concurrent trigeminal measurement provides an inherent system check.

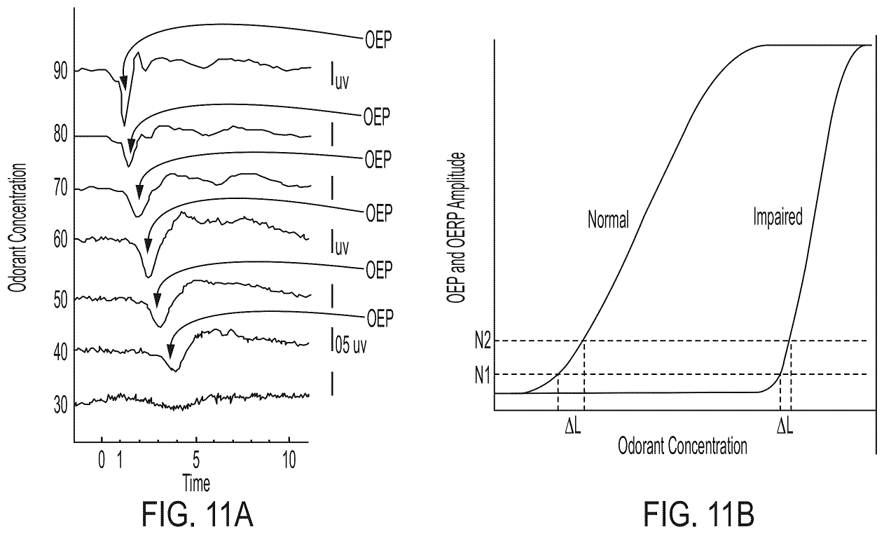

According to some embodiments of the present invention, and as illustrated in FIGS. 11A and 11B, a “staircase” procedure may be utilized to estimate the neural threshold for eliciting an OEP from a subject or to estimate that subject's odor sensitivity. The concentration staircase is an approach commonly used in measuring auditory evoked potentials. For example, FIGS. 11A and 11B present a hypothetical example Illustrating the effects of increasing odorant concentration on the amplitude of olfactory on evoked potentials.

Changes in olfactory evoked potential amplitude with increases in odorant concentration (bottom to top waveforms) are illustrated in FIG. 11A. At the lowest concentration, the evoked potential is not obvious, but by repeating the evoked potential with increasing odorant concentration, the evoked potential response becomes apparent. When the growth in the amplitude of the evoked potential (FIG. 11B) is compared in the same patient over time or across patients, it is possible to identify impaired olfactory sensitivity (i.e., when the head is struck with force, the skull moves immediately but there is a phase lag in the response of the brain, which can cause olfactory neurons passing from the olfactory epithelium through the cribriform plate to the olfactory bulb to be sheared decreasing sensitivity). Embodiments of the present invention utilize a similar procedure such that, by repeating the OEP with increasing odorant concentrations, changes in function that might serve as a biomarker for neurological insult can be identified.

According to some embodiments of the present invention, the odorant concentration from OEP to OEP will be increased from, for example, 10%, then 20%, then 30% . . . up to 100%. The OEP will be measured at each concentration. For the lowest odorant concentrations, the OEP may be too small, and may not be visible over the neural background noise floor. However, as the odorant concentration is increased, the OEP signal amplitude will grow and become visible above the noise floor. Measurement continues to 100% odorant concentration, even if the OEP peak is observed at much lower concentrations, and the higher level peaks can be used to verify the lower, near threshold, smaller peaks. Threshold can be defined in many ways, such as the first odorant concentration where the OEP peak is 0.5 μV above the noise floor.

FIG. 11B illustrates hypothetical changes in neural responding with odorant concentration. The top trace illustrates an OEP at odorant concentrations, and lower traces with progressive decreases in odorant concentration. The lower the odorant concentration that an OEP can be observed, the more sensitive the nose. Over time, or with neurological insult, higher odorant concentrations, indicating a decrease in olfactory sensitivity, are required to evoke the same amplitude OEP/OERP.

In general, OERP amplitude is not sensitive to odorant level, or in some embodiments OERPs will include measuring OERPs using odorant pulses of the same concentration.

Auditory sound stimuli and somatosensory vibrotactile stimuli can be of a fixed, or varied amplitude.

FIG. 5 illustrates an odorant generator 100, an auditory generator 330, and a somatosensory generator 340 according to some embodiments of the present invention. In the illustrated embodiment, the odorant generator 100 includes two valves 102a, 102b which control the airflows that create the “odorant pathway” channel. Ambient air may be drawn through a desiccator 104 and a charcoal filter 106 via an air pump 107 that is in fluid communication with the valves 102a, 102b. The illustrated embodiment of the odorant generator 100 also includes a passive odorant cartridge 108 and odorant proportional valves 110a, 110b. Valve 102a controls the airflow through the passive odorant cartridge 108 to produce a 100% saturated odorant, and forms the input to the odorant proportional valves 110a, 110b for mixing with the clean air dilution pathway from valve 102b. The passive odorant cartridge 108 is used to aerosolize the odorant and create a saturated airspace. The output from the of the passive odorant cartridge 108 is 100% saturated airflow. In an additional embodiment, the cartridge is replaced by a nebulizer/ultrasonicator. The odorant concentration generated may not be 100% so long as the concentration is verifiable via a photo ionization detector, CMOS or the like, and repeatable from use to use.

In the illustrated embodiment, the two proportional valves 110a, 110b are used to produce variable specified odorant dilutions. Valve 110a is in fluid communication with the passive odorant cartridge 108, and valve 110b is in fluid communication with clean air for diluting the odorant to a target odorant concentration. In some embodiments of the present invention, both valves 110a, 110b may be mounted in a manifold 112.

Using the illustrated two proportional valve arrangement, any odorant concentration from 0 to 100% can be produced. The two valves 110a, 110b control the release of the saturated odorant and air, respectively, and cause mixing of the odorant with the filtered air in the right proportions to create the desired target concentration. In some embodiments of the present invention, a small mixing space may be utilized to make sure that an odorant is thoroughly mixed into and diluted by the clean-air. These proportional valves 110a, 110b may be controlled, for example, using a variable DC control signal.

In some embodiments, an odorant utilized by the odorant generator 100 can comprise a gel odorant wherein the gel is held in a mesh/perforated structure, or in a polymer that can be released within a cartridge. In some embodiments, a liquid phase odorant (e.g., from ampoules) may be dispensed, prior to use/on cartridge insertion, onto an absorbent diaper-like material. In some embodiments, a multiple reservoir cartridge that holds two or three different dilutions of an odorant could be utilized.

Still referring to the embodiment illustrated in FIG. 5, a 3-way valve 202 takes the target odorant in the specified dilution from the manifold 112 and/or proportional valves 110a, 110b as one input, and clean, filtered air directly from the air pump 107 as the second input. The flow rate of both the odorant and clean-air stream may be the same (e.g., about 8-10 liter per minute). The 3-way valve 204 can release a continuous stream from the filtered air except when the target odorant pulse is to be delivered, when it will switch and deliver the target odorant for 200 to 1000 milliseconds, then switch back and deliver the clean, filtered air flow. The switching from one to the other, and vice versa, may be on the order of about 1 to 5 milliseconds, for example, and should result in as close as possible to a square wave form as can be achieved with airflows.

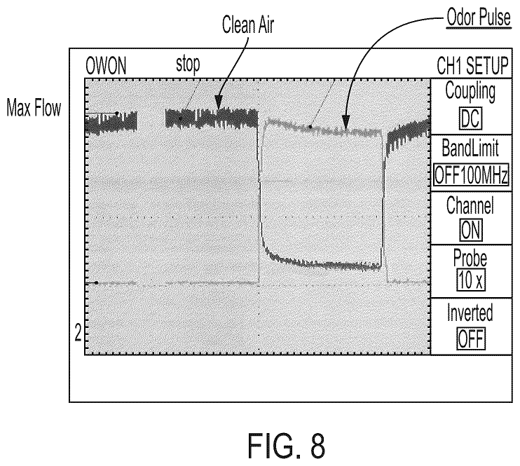

FIG. 8 illustrates a measured, exemplary square wave form where an odorant channel is presented into a gap where the fresh air channel has been gated off, creating an odorant pulse duration of 600-800 ms without an overall change in air pressure detectable by a human observer.

Valves 202, 204 allow the delivery of a stimulus (i.e., odorant) embedded in a constantly flowing air stream such that subjects do not perceive the switching from odorless to odorized air. Subjects receive a constant intranasal airflow (e.g., about 6 liter/minute) which is humidified (e.g., about 80% relative humidity) and warmed to body temperature (e.g., about 36° C.) such that, following a short period of adaptation, administration of the constant airflow is not perceived by the subject.

Still referring to FIG. 5, test subject interface components 330 and 340 complete the multisensory system, according to some embodiments of the present invention. Auditory sound stimuli created by a speaker under the direction of the controller 120 is communicated to the test subject's ear by earbud transducers inserted into the external ear canal, or by an acoustic headphone speaker placed near the ear, etc. Sound stimuli may be delivered at a moderate (˜70 dB SPL) level, although various decibel levels may be utilized. Somatosensory stimuli are created by a vibrotactile stimulator (e.g., a vibration device attached to the body, such as the back of the hand, etc.) and under control by the controller 120. Somatosensory evoked potentials can be evoked by vibrotactile stimulator, or a 0.2-2 millisecond duration electrical stimulus, delivered to surface electrodes on the medial nerve at the wrist. Tactors, such those as available from Engineering Acoustics, Inc., Casselberry, Florida, can be attached to the finger tips, and can stimulate at 60 Hz, or a single square pulse.

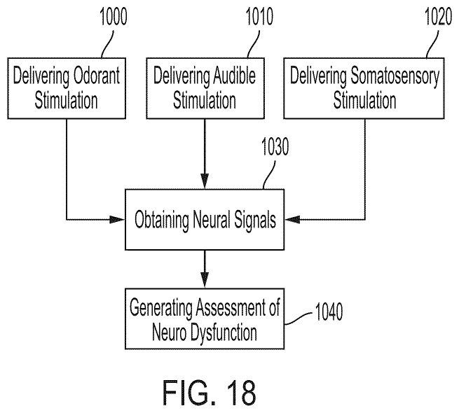

FIG. 18 is a flow chart illustrating methods of measuring neurologic function according to some embodiments of the present invention. One or more of an odorant stimulation (Block 1000), an audible stimulation (Block 1010), and a somatosensory stimulation (Block 1020) may be delivered to a subject. In some embodiments, the odorant stimulation, the audible stimulation, and the somatosensory stimulation are delivered to the subject sequentially. In some embodiments, the odorant stimulation, the audible stimulation, and the somatosensory stimulation are delivered to the subject substantially simultaneously. In some embodiments, the audible stimulation and the somatosensory stimulation are delivered to the subject before the odorant stimulation. In some embodiments, the audible stimulation and the somatosensory stimulation are delivered to the subject after the odorant stimulation. The odorant stimulation, audible stimulation, and somatosensory stimulation cause the generation of neural signals from the subject, and these neural signals are obtained (Block 1030) via electrodes attached to the subject, e.g., attached to the scalp, etc. The neural signals are processed by one or more processors to generate an assessment of neurologic function of the subject. An assessment of neurologic dysfunction of the subject, such as from mTBI or a concussion, can then be determined by comparing the generated neurologic function assessment to a baseline of neurologic function for the subject. (Block 1040). In some embodiments, a determination of the presence of a neurodegenerative disease, the increased likelihood of eventual onset of a neurodegenerative disease, or the presence of mTBI can be judged by comparison of a single neurological function to a demographic population database of similar olfactory evoked potentials.

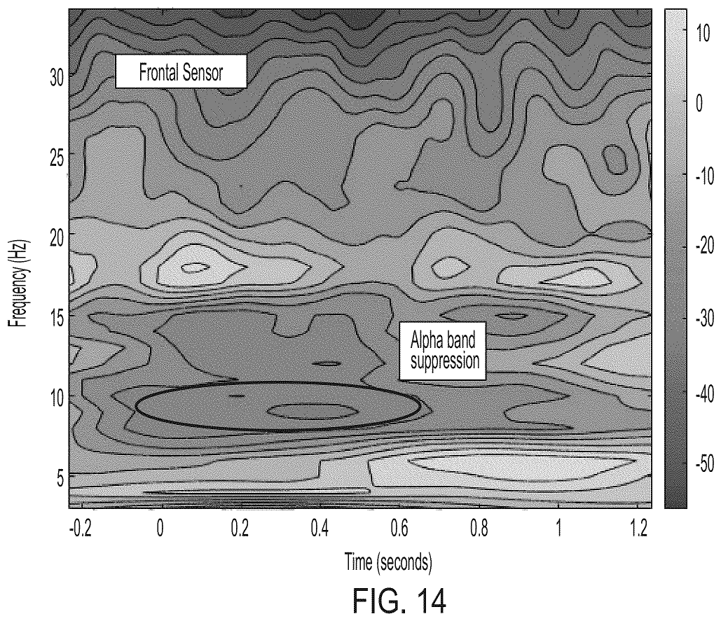

The neural signals can be visualized using quantitative electroencephalography (qEEG). Sensory cortical evoked potentials (e.g., olfactory, auditory and/or somatosensory) can be viewed directly as voltage waveforms, or indirectly as changes in brain oscillations (e.g., alpha, beta, gamma, theta, etc.) as shown in FIG. 14.

According to embodiments of the present invention, sensory neural activity evoked by odorant, auditory and/or somatosensory stimuli can be measured from electrodes, e.g., scalp electrodes. After recording the evoked neural responses, the neural responses can be measured directly or by their effect on other brain responses, such as beta, theta and/or alpha band oscillations using qEEG. For example, when odorants are presented to the nose, they produce significant suppression or desynchronization of alpha band oscillations.

Concussions and mTBI can interfere with alpha band desynchronization produced by working memory tests. Working memory tests are typified by asking a person to repeat a sequence of numbers, then asking them to recall the number x or y before the last number. Working memory tests are behavioral and require active participation from the subject. These are affected by attention, education, language, cooperation, etc., or variations in test conditions or examiner expertise. Embodiments of the present invention generate evoked sensory responses and do not require cooperation from the subject.

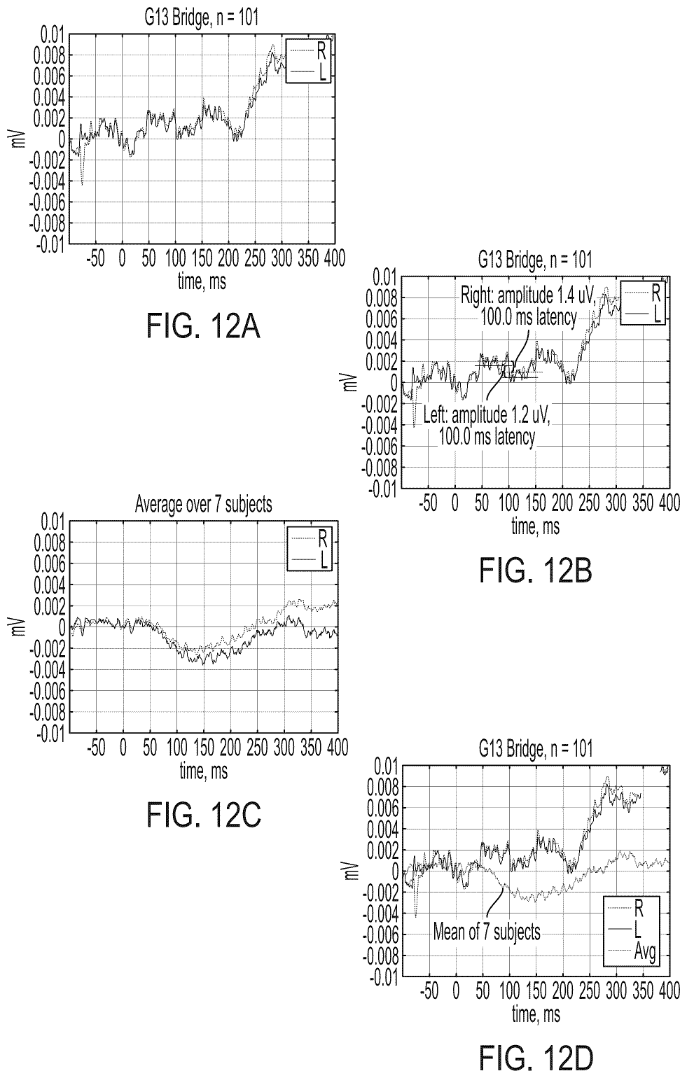

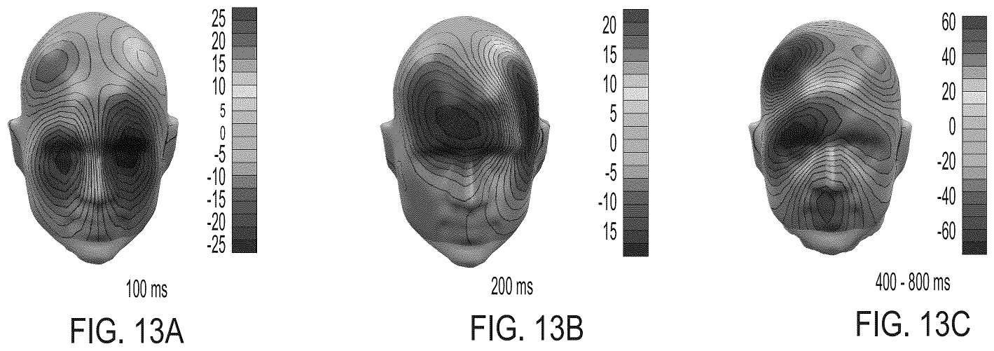

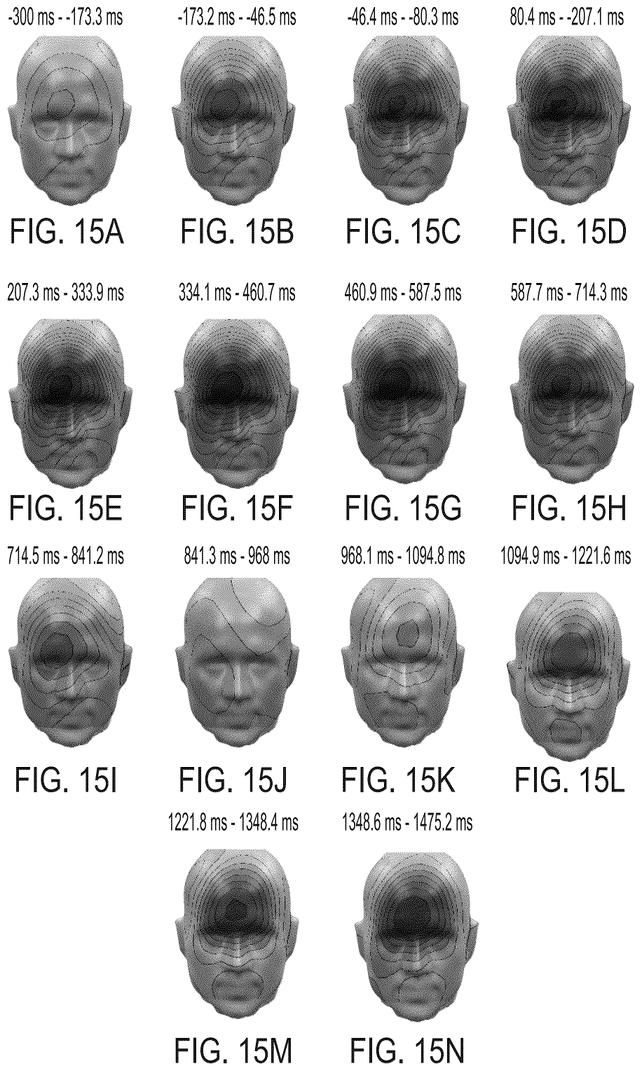

Concussions and mTBI can be identified as either baseline-post concussion comparisons of evoked responses (e.g., decreases in amplitude of waveforms as shown in FIG. 12A-12D), decreases in spread or amplitude of voltage gradients (e.g. as shown in FIG. 13), changes in the speed or location of current flow from one brain location to another (i.e., current might not flow between two normal brain cortices if there is mTBI brain damage in between; see FIG. 15—where damage might prevent flow between 15I and 15J), or the alpha band suppression present in the oval in FIG. 14 might be lost. The same or similar changes caused by the slower deterioration due to neurologic disease may be measurable, albeit over a longer time period (slow progression of a neurodegenerative disease), hence a measure of “neurologic dysfunction.”

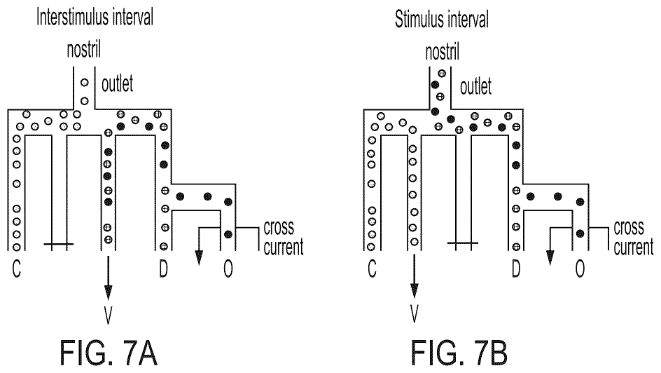

Referring to FIGS. 7A-7B, in order to embed brief odor pulses within constant air flow, two airstreams are produced. One contains odorless control air (C) whereas the other contains an odorant (O) at a defined concentration. Different odor concentrations may be obtained by adjusting the dilution (D) of the odorant. A vacuum system (V) may be utilized, as well as a small cross current of odorless air, which prevents molecules of the (O) tubing from being drawn into other tubes.

During the interstimulus interval (FIG. 7A), a subject's nose receives control air only. The odorized (O+D) airstream is directed towards the vacuum (V) line and directed to the charcoal filter. During stimulation, the (O+D) airstream is briefly directed to the outlet of the stimulator, and control air is redirected towards the vacuum line (V) (FIG. 7B). Using this system, it is therefore possible to switch between an odorless and an odorized air stream in less than about 10 to 20 milliseconds. Airflow rates are calibrated and the stimulus and no-stimulus conditions are controlled by the valves 202, 204 described above in FIG. 5. Subjects do not perceive the control air which is presented during the interstimulus interval and the switching between odorless and odorized air is not accompanied by any mechanical or thermal changes in the airflow. However, it should be noted that subjects will feel the control air, just not changes/perturbations in the flow rate of the control air. If the flow rate remains constant, the mechanoreceptors will adapt and will not produce an electrical response that might modify the olfactory waveform. It is critical that the only changes in stimulation detected be the onset of the odorant by the olfactory receptors.

The various valves of the odorant generator 100 and the valve assembly 200 illustrated in FIGS. 1 and 5 are controlled by a controller 120, such as an Intel NUC, Arduino, or a Raspberry Pi controller. The controller 120 takes a USB input from a computer 500 (FIG. 1) and triggers the odorant pulses. The controller 120 stores all stimulus information, including but not limited to, odorant duration, odorant concentration, inter-odor pulse interval, etc. The final odorant line valve 202 can send a voltage signal to the computer to verify precisely when the odorant pulse is delivered to the test subject.

The computer 500 (FIG. 1) or the controller 120 (FIG. 5) may have software for performing signal averaging and signal processing to computationally increase the OEP/OERP waveform signal-to-noise ratio and aid in response detection, calculating of OEP/OERP response latency and amplitude. The computer 500 may also store subject Hx, responding and analytics. The computer 500 may also run software to analyze EEG waveforms utilizing standard software programs such as MatLAB (Mathworks, Natick, MA) EEGLab.

A vacuum pump 130 (FIG. 5) can be used to draw residual odorants through a charcoal filter 140, and limit contamination to and from the intranasal delivery assembly 300. The vacuum pump 130 can also be configured to assist in the creation of the odorant pulses with the three-way valves 202, 204.

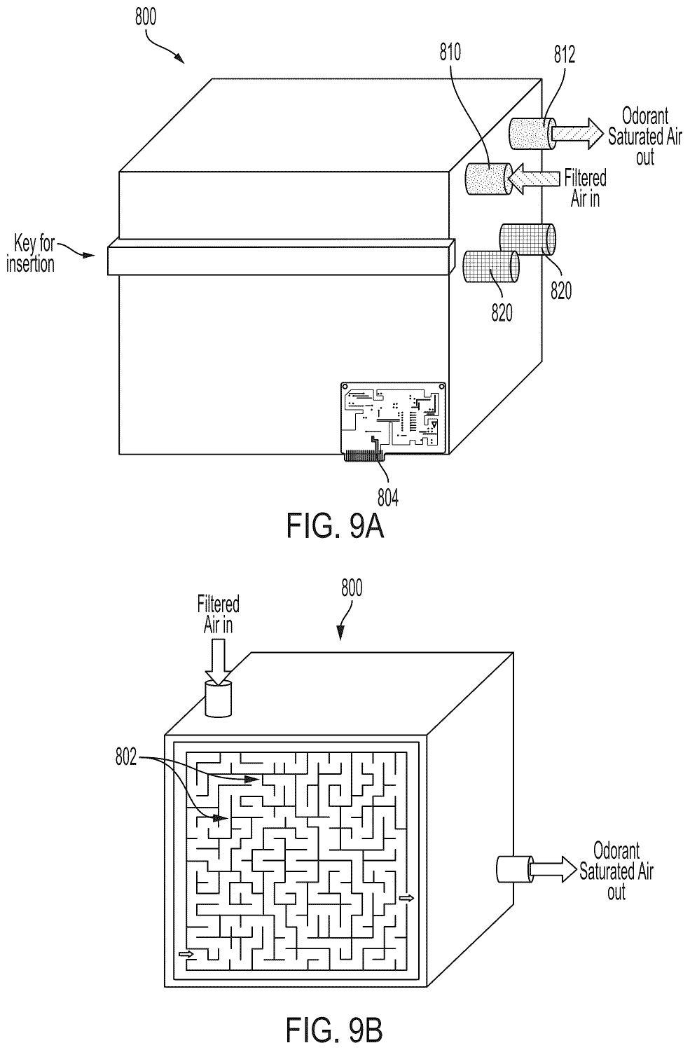

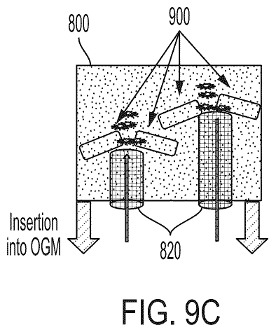

Referring now to FIGS. 9A and 9B, an odorant cartridge 800 which is configured to rapidly aerosolize an odorant and which can be utilized as the passive odorant cartridge 108 in FIG. 5, is illustrated. The illustrated cartridge 800 is a reservoir for creating/storing saturated odorant. The cartridge 800 can contain a liquid phase odorant. The cartridge protects against leakage and oxidation.

The illustrated cartridge 800 includes a filtered air inlet port 810 and an odorant saturated air outlet port 812, and the inlet port 810 and outlet port 812 are located on the same side of the cartridge 800. These ports 810, 812 may be capable of an airtight seal and of being punctured when the cartridge 800 is inserted into a receiving assembly/device of the odorant generator 100, allowing in flow of clean, filtered air, and out flow of saturated odorant. The ports 810, 812 may be high on the side of the cartridge 800 to prevent any possible liquid leaking liquid odorants that might be accumulating on the floor of the cartridge 800.

The illustrated cartridge 800 includes plungers 820 that are forced inwardly when the cartridge 800 is inserted in a receiving assembly/device of the odorant generator 100. The plungers 820 are configured to mechanically break ampoules 900 (FIG. 9C), such as glass ampoules, within the cartridge 800 to release liquid phase odorant onto the absorbent material in the interior 802 of the cartridge 800.

The volume of the cartridge 800 may also serve as a reservoir for the saturated gas phase odorant. If, for example, the cartridge 800 has a volume of 250 milliliters (ml), it might hold a sufficient volume of saturated gas phase odorant to create hundreds of stimulates at relatively low concentrations.

As illustrated in FIG. 9B, the inside of the cartridge includes a maze-like structure 802 or a plurality of louvers 802, which significantly increase the surface area. The interior structure of the cartridge 800 may be constructed of a wicking or absorbent material configured to hold and disburse a liquid phase odorant. In some embodiments, the internal volume of the cartridge is between about 80-320 milliliters and serves as a reservoir for multiple odorant stimuli. The odorant passes from the liquid phase to a gas phase through diffusion. The illustrated cartridge 800 includes an external circuit board 804 and connection so that the cartridge 800 can be controlled and powered by the computer 500 (FIG. 1). With the power from the computer 500, a coil may be heated or an internal ultrasonicator may be activated to aid in aerosolization. The cartridge 800 could be single use and disposable, or multi use.

According to other embodiments of the present invention, the cartridge 800 can have multiple spaces for different odorants or the same odorant in different concentrations.

According to other embodiments of the present invention, the cartridge 800 can have a powered agitator or whisk to move the odorants around and aid in aerosolization.

According to other embodiments of the present invention, the cartridge 800 can have a ultrasonicator/nebulizer to facilitate in aerosolization.

According to other embodiments of the present invention, the cartridge 800 can have a heating filament for maintaining a desired temperature of the odorant to facilitate aerosolization.

Referring back to FIG. 1, exemplary signal amplifiers and signal amplifiers that may be utilized as the signal amplifier/processor 400 are available from Tucker-Davis Technologies, Alachua, Florida. Exemplary software for controlling the system 10 of FIG. 1, including signal processing and display via the display on computer 500, is also available from Tucker-Davis Technologies. In some embodiments of system 10, the exemplary signal amplifier/processor 400 and computer 500 may be enclosed in the odorant generator 100 case as opposed to being externally connected. Other sources for equipment and software that may be utilized in accordance with embodiments of the present invention are available from, for example, Cambridge Research Systems, Rochester, United Kingdom; Biopac Systems, Goleta, California; Stoelting, Inc., Wood Dale, Illinois; World Precision Instruments, Sarasota, Florida; Thomas Recording GmbH, Giessen, Germany; Lafayette Instrument Company, Lafayette, Indiana; Scientifica, Uckfield, United Kingdom; Emotiv Inc., San Francisco, California; Imotions, Boston, Massachusetts; Neurosky, San Jose, California; Multi Channel Systems MCS GmbH, Reutlingen, Germany.

FIGS. 12A-12D, 13A-13C, 14, 15A-15N, 16A-16B and 17A-17F are exemplary reports generated using data collected by embodiments of the present invention. FIG. 12A-12D illustrate exemplary OEP individual wave form data that can be obtained using the system of FIG. 1. FIG. 12A illustrates hypothetical raw OEP waveforms. FIG. 12B illustrates hypothetical OEP waveforms with measurement guides. FIG. 12C illustrates hypothetical group mean OEP waveforms for each nostril of a nose. FIG. 12D illustrates hypothetical group grand mean OEP (averaged across nostril) waveforms.

FIGS. 13A-13C illustrate exemplary OERP individual topographical heat map data that can be obtained using the system of FIG. 1. The topographic heat maps show voltage gradients at different times post odorant activation for a hypothetical subject, superimposed on a standard head. At 100 ms (FIG. 13A), the calculated OEP onset latency, the red zones indicates activation of the olfactory epithelium. With increases in latency from 100 ms to 400-800 ms, the site of activation (shades of red) indicate a shift in activation to deeper, more central neural sites, likely olfactory bulb and piriform cortex.

FIG. 14 illustrates exemplary alpha wave oscillation data obtained using the system of FIG. 1. Alpha wave oscillation occurs when the brain is idling, resting, i.e., when unstimulated. When a stimulus is presented (odorant, auditory or vibrotactile), the brain is excited and the idling (alpha) oscillation is suppressed or desynchronized because the brain is now attending to the new (odorant, auditory or vibrotactile) stimulus. The alpha oscillation was largest between 8 and 9 Hz, and a significant negativity (shades of blue within the red circled alpha frequency band) indicate a suppression, or desynchronization, of alpha responding beginning just prior to odorant delivery. The alpha desynchronization likely preceded odorant onset due to the sounds generated by the activation of the odorant delivery valves.

FIGS. 15A-15N illustrate exemplary alpha wave spectra grand mean data from a group of hypothetical subjects tested using the system of FIG. 1. Illustrated are topographic voltage gradient distribution of alpha wave spectra projected onto a standard head, with time course of changes following odorant onset, grand mean average of six hypothetical subjects. Greater odorant ERP suppression in alpha peak amplitude is shown in shades of blue, and red shows an increase in alpha after odorant cessation. Indicated time epochs have been affected by the analysis procedure.

The alpha wave is present when the brain is idling and alert (it is thought to play a role in attention), but is suppressed when the brain is stimulated, in this case, by the odorant. Recent studies have recently shown that this event-related change in alpha activity (e.g., shown between the topographic maps −300 ms-173.3 and −173.2-−46.5; and from 714.5 ms-841.2 ms to 968.1 ms-1094.8 ms) is a sensitive biomarker for concussions (c.f., Arakaki et al., 2018; Guay et al., 2018, which are incorporated herein by reference in their entireties).

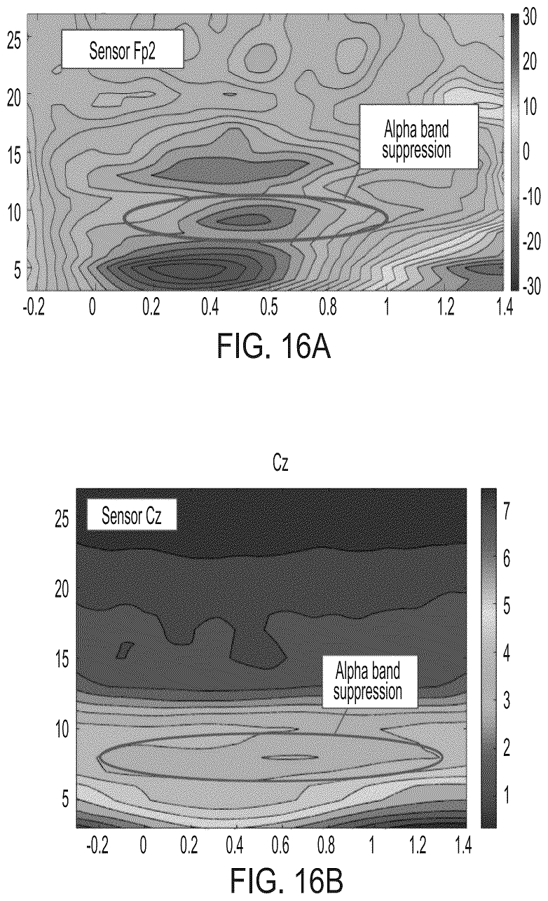

FIGS. 16A-16B illustrate exemplary alpha band suppression grand mean data from a group of hypothetical subjects tested using the system of FIG. 1. Examples of alpha suppression at different time points following odorant stimulus onset. FIG. 16A illustrates response at an electrode on the front of the head, where the alpha oscillation was largest (note difference in scales for FIGS. 16A and 16B). In FIG. 16A, a significant negativity (shades of blue within the red circled alpha frequency band) indicate a decrease in responding beginning just prior to odorant delivery. The alpha suppression likely preceded odorant onset due to the sounds generated by the activation of the odorant delivery valves.

A suppression of the alpha response (frequency ˜9 Hz), though much smaller, is also observed in FIG. 16B for sensor Cz, on the vertex of the skull. Though smaller, the alpha suppression is demonstration of the alpha oscillation (prior to stimulus onset).

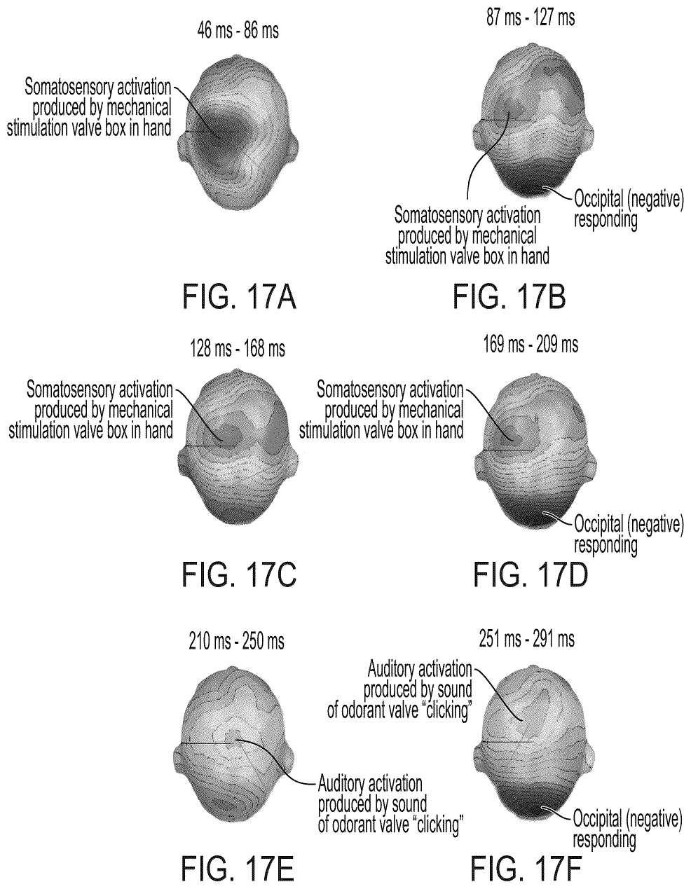

FIGS. 17A-17F illustrate exemplary multisensory data using the system of FIG. 1. Locations of somatosensory and auditory activation voltage gradients (shades of red/yellow) are superimposed on a standard head model. Auditory and vibrotactile stimuli were activated simultaneously. Activation is located over auditory and somatosensory cortices.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

That which is claimed is:

1. A system for measuring neurologic function of a subject, the system comprising:
an odorant generator configured to deliver an odorant stimulation to the subject;
an auditory generator configured to deliver an audible stimulation to the subject;
at least one electrode configured to be attached to the subject, wherein the at least one electrode is configured to collect neural signals from the subject as a result of the odorant stimulation and the audible stimulation; and
at least one processor configured to process the neural signals from the at least one electrode and generate an assessment of neurologic function of the subject.

2. The system of claim 1, further comprising a vibrotactile stimulator configured to generate a somatosensory stimulation to the subject, and wherein the at least one electrode is configured to collect neural signals from the subject as a result of the somatosensory stimulation.

3. The system of claim 1, wherein the at least one electrode comprises a plurality of electrodes configured to be attached to the subject at respective different locations.

4. The system of claim 1, wherein the auditory generator is configured to deliver an audible stimulation to the subject via one or more earbuds worn by the subject.

5. The system of claim 2, wherein the vibrotactile stimulator is configured to generate a somatosensory stimulation to skin of the subject.

6. The system of claim 1, wherein the odorant generator comprises a handheld intranasal delivery assembly or a mask configured to be placed over a face of the subject.

7. A system for measuring neurologic function of a subject, the system comprising:
an odorant generator configured to deliver an odorant stimulation to the subject;
an auditory generator configured to deliver an audible stimulation to the subject;
a vibrotactile stimulator configured to generate a somatosensory stimulation to the subject;
a plurality of electrodes configured to be attached to the subject at respective different locations, wherein the plurality of electrodes are configured to collect neural signals from the subject as a result of the odorant stimulation, the audible stimulation, and the somatosensory stimulation; and
at least one processor configured to process the neural signals from the plurality of electrodes and generate an assessment of neurologic function of the subject.

8. The system of claim 7, wherein the auditory generator is configured to deliver an audible stimulation to the subject via one or more earbuds worn by the subject, and wherein the vibrotactile stimulator is configured to generate a somatosensory stimulation to skin of the subject.

9. The system of claim 7, wherein the odorant generator comprises a handheld intranasal delivery assembly.

10. A method of measuring neurologic function of a subject, the method comprising:
delivering an odorant stimulation to the subject;
delivering an audible stimulation to the subject;
delivering a somatosensory stimulation to the subject;
collecting neural signals from the subject via one or more electrodes attached to the subject as a result of the odorant stimulation, the audible stimulation, and the somatosensory stimulation; and
processing the neural signals via at least one processor to generate an assessment of neurologic function of the subject.

11. The method of claim 10, wherein the odorant stimulation, the audible stimulation, and the somatosensory stimulation are delivered to the subject substantially at the same time.

12. The method of claim 10, wherein the odorant stimulation, the audible stimulation, and the somatosensory stimulation are delivered to the subject sequentially.

13. The method of claim 10, wherein the audible stimulation and the somatosensory stimulation are delivered to the subject before the odorant stimulation.

14. The method of claim 10, wherein the audible stimulation and the somatosensory stimulation are delivered to the subject after the odorant stimulation.

15. A system for measuring neurologic function of a subject, the system comprising:
an odorant generator configured to deliver an odorant stimulation to the subject;
at least one electrode configured to be attached to the subject, wherein the at least one electrode is configured to collect neural signals from the subject as a result of the odorant stimulation; and
at least one processor configured to process the neural signals from the at least one electrode and generate an assessment of neurologic function of the subject.

16. The system of claim 15, wherein the at least one electrode comprises a plurality of electrodes configured to be attached to the subject at respective different locations.

17. The system of claim 15, wherein the odorant generator comprises a handheld intranasal delivery assembly or a mask configured to be placed over a face of the subject.
